(19) United States
(12) Patent Application Publication
(10) Pub. No.: US 2021/0045870 A1
(43) Pub. Date: Feb. 18, 2021

(54) REPLACEMENT HEART VALVE LOCKING ELEMENT

(71) Applicant: BOSTON SCIENTIFIC SCIMED, INC., MAPLE GROVE, MN (US)

(72) Inventor: Joel N. Groff, Delano, MN (US)

(73) Assignee: BOSTON SCIENTIFIC SCIMED, INC., MAPLE GROVE, MN (US)

(21) Appl. No.: 16/991,563

(22) Filed: Aug. 12, 2020

Related U.S. Application Data
(60) Provisional application No. 62/886,561, filed on Aug. 14, 2019.

(51) Int. Cl. A61F 2/24

(52) U.S. Cl. CPC A61F 2/2418; A61F 2220/0016; A61F 2/2427

(57) ABSTRACT

A locking element for securing an expandable anchor member in a shortened configuration may include a plurality of engagement features configured to releasably secure the locking element to a first end portion of the expandable anchor member without using any external elements, wherein the locking element is configured to engage a post member secured to a second end portion of the expandable anchor member opposite the first end portion in the shortened configuration.

### CROSS-REFERENCE TO RELATED APPLICATIONS

This application claims the benefit of priority of U.S. Provisional Application No. 62/886,561 filed Aug. 14, 2019, the entire disclosure of which is hereby incorporated by reference.

### TECHNICAL FIELD

The present disclosure pertains to medical devices, and methods for manufacturing and/or using medical devices. More particularly, the present disclosure pertains to an improved design for a locking element for a replacement heart valve.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In a first aspect, a locking element for securing an expandable anchor member in a shortened configuration may comprise a plurality of engagement features configured to releasably secure the locking element to a first end portion of the expandable anchor member without using any external elements. The locking element may be configured to engage a post member secured to a second end portion of the expandable anchor member opposite the first end portion in the shortened configuration.

In addition or alternatively, the expandable anchor member surrounds at least a portion of one or more of the plurality of engagement features when the locking element is releasably secured to the first end portion of the expandable anchor member.

In addition or alternatively, the locking element includes a post passageway configured to axially receive the post member.

In addition or alternatively, the locking element includes a plate portion extending from a first end of the locking element to a second end of the locking element.

In addition or alternatively, at least a portion of the plate portion engages an inner surface of the expandable anchor member when the locking element is releasably secured to the first end portion of the expandable anchor member.

In addition or alternatively, at least a portion of the plurality of engagement features engages an outer surface of the expandable anchor member when the locking element is releasably secured to the first end portion of the expandable anchor member.

In addition or alternatively, at least a portion of the plurality of engagement features extends outward through the expandable anchor member when the locking element is releasably secured to the first end portion of the expandable anchor member.

In addition or alternatively, the plate portion includes a groove extending from the first end of the locking element to a second end of the locking element.

In addition or alternatively, each of the plurality of engagement features extends laterally from the plate portion.

In another aspect, a replacement heart valve implant may comprise an expandable anchor member configured to shift between an elongated configuration and a shortened configuration, the expandable anchor member having a first end portion and a second end portion opposite the first end portion; a locking element releasably secured to the first end portion of the expandable anchor member by a plurality of engagement features of the locking element without using any external elements; and a post member secured to the second end portion. The locking element may be configured to secure the expandable anchor member in the shortened configuration when engaged with the post member.

In addition or alternatively, the locking element includes a tongue portion configured to releasably attach the locking element to an implant delivery device.

In addition or alternatively, at least a portion of the plurality of engagement features is disposed outward of the expandable anchor member.

In addition or alternatively, the post member is coupled to a plurality of valve leaflets disposed within the expandable anchor member.

In addition or alternatively, the expandable anchor member includes a loop at the first end portion, the loop engaging at least one of the plurality of engagement features.

In addition or alternatively, the plurality of engagement features prevents the locking element from disengaging from the expandable anchor member without manual manipulation.

In another aspect, a locking element for securing an expandable anchor member in a shortened configuration may comprise a plate portion, a tongue portion extending axially from the plate portion, and a plurality of engagement features extending laterally from the plate portion in a first direction. The plurality of engagement features may comprise a head portion, a first arm portion, a second arm portion, and a flap portion. The plurality of engagement features may be configured to releasably secure the locking element to a first end portion of the expandable anchor member. The flap portion may be configured to engage a post member secured to a second end portion of the expandable anchor member opposite the first end portion in the shortened configuration. The locking element may be releasably securable to the first end portion of the expandable anchor member without any other structure.

In addition or alternatively, the head portion includes a head offset extending generally parallel to the plate portion.

In addition or alternatively, the first arm portion includes a first arm offset extending generally parallel to the plate portion and the second arm portion includes a second arm offset extending generally parallel to the plate portion.

In addition or alternatively, the plate portion includes an axially-oriented groove formed in the plate portion and opening out in the first direction, the axially-oriented groove being configured to receive at least a portion of the post member when the expandable anchor member is in the shortened configuration.

In addition or alternatively, the head portion, the first arm portion, the second arm portion, and the flap portion are configured to extend exterior of the expandable anchor member when the locking element is releasably secured to the first end portion of the expandable anchor member with the plate portion disposed within the expandable anchor member.

The above summary of some embodiments, aspects, and/or examples is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an example medical device system;

FIG. 2 illustrates an example replacement heart valve implant associated with the example medical device system;

FIG. 3 is a perspective view illustrating aspects of a locking element associated with the example replacement heart valve implant;

FIG. 4 is a front view illustrating aspects of the locking element shown in FIG. 3;

FIG. 5 is a side view illustrating aspects of the locking element shown in FIG. 3;

FIG. 6 is bottom/end view illustrating aspects of the locking element shown in FIG. 3;

FIG. 7 is a perspective view illustrating aspects of an expandable anchor member associated with the example replacement heart valve implant;

FIG. 8 illustrates aspects of the locking element of FIG. 3 secured to the expandable anchor member of FIG. 7 as seen from outside the expandable anchor member; and

FIG. 9 illustrates aspects of the locking element of FIG. 3 secured to the expandable anchor member of FIG. 7 as seen from inside the expandable anchor member.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some relatively common medical conditions may include or be the result of inefficiency, ineffectiveness, or complete failure of one or more of the valves within the heart. For example, failure of the aortic valve or the mitral valve can have a serious effect on a human and could lead to a serious health condition and/or death if not dealt with properly. Treatment of defective heart valves poses other challenges in that the treatment often requires the repair or outright replacement of the defective heart valve. Such therapies may be highly invasive to the patient. Disclosed herein is a medical device system that may be used for delivering a medical implant to a portion of the cardiovascular system in order to diagnose, treat, and/or repair the system. At least some of the medical implants and/or systems disclosed herein may be used to deliver and implant a replacement heart valve implant (e.g., a replacement aortic valve, replacement mitral valve, etc.). In addition, the medical device system may deliver the replacement heart valve implant percutaneously and, thus, may be much less invasive to the patient. The device and/or system disclosed herein may also provide other desirable features and/or benefits as described below.

The figures illustrate selected components and/or arrangements of a medical device system 10. It should be noted that in any given figure, some features of the medical device system 10 may not be shown, or may be shown schematically, for simplicity. Additional details regarding some of the components of the medical device system 10 may be illustrated in other figures in greater detail. A medical device system 10 may be used to deliver and/or deploy a variety of medical devices to a number of locations within the anatomy. In at least some embodiments, the medical device system 10 may include an implant delivery device (e.g., a replacement aortic valve delivery device) that can be used for percutaneous delivery of a replacement heart valve. This, however, is not intended to be limiting as the medical device system 10 may also be used for other interventions including mitral valve replacement, valve repair, valvuloplasty, and the like, or other similar interventions.

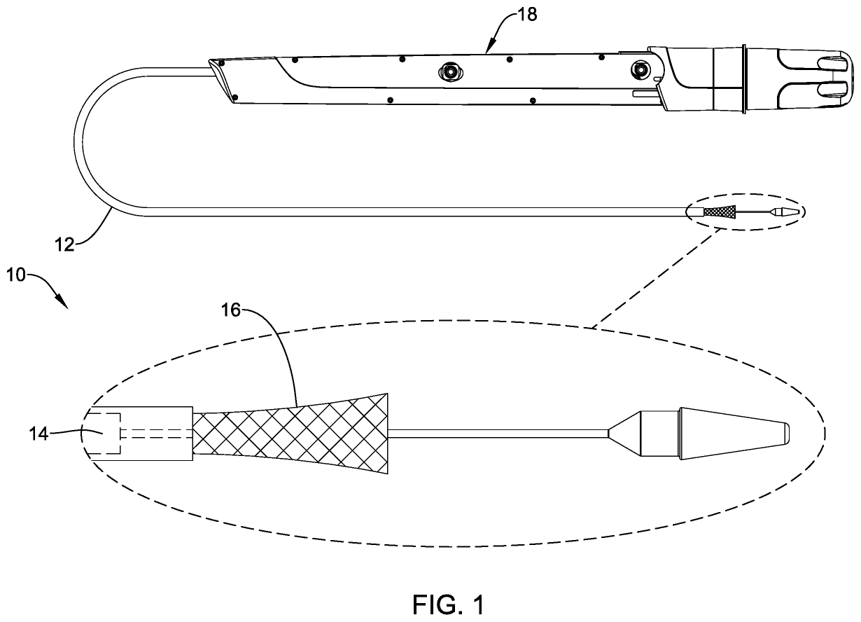

The medical device system 10, as seen in FIG. 1 for example, may generally be described as a catheter system that includes an implant delivery device having an outer sheath 12 for a replacement heart valve implant 16 which may be coupled to the implant delivery device and disposed within a lumen of the outer sheath 12 during delivery of the replacement heart valve implant 16. In some embodiments, the implant delivery device may include an inner catheter 14 extending at least partially through the outer sheath 12 (partially seen in phantom in FIG. 1). In some embodiments, the replacement heart valve implant 16 may be coupled to the inner catheter 14 and disposed within the lumen of the outer sheath 12 during delivery of the replacement heart valve implant 16. In some embodiments, a handle 18 may be disposed and/or attached at a proximal end of the implant delivery device, as seen in FIG. 1, and may include one or more actuation means associated therewith. In some embodiments, the handle 18 may have an outer shell and an interior space. In some embodiments, the handle 18 may have a control knob rotatable relative to the outer shell of the handle 18. In some embodiments, the handle 18 and/or the control knob may be configured to manipulate the position of the outer sheath 12 relative to the inner catheter 14 and/or the replacement heart valve implant 16, and/or to aid in the deployment of the replacement heart valve implant 16. In some embodiments, the medical device system 10 may include a nose cone disposed at a distal end of a guidewire extension tube, wherein the guidewire extension tube may extend distally from the inner catheter 14. The medical device system 10 and/or the guidewire extension tube may be configured to slidably receive and/or slidably move over a guidewire. In at least some embodiments, the nose cone may be designed to have an atraumatic shape. In some embodiments, the nose cone may include a ridge or ledge that is configured to abut a distal tip of the outer sheath 12 during delivery of the replacement heart valve implant 16. Some suitable but non-limiting materials for the medical device system 10, implant delivery device, the outer sheath 12, the inner catheter 14, the handle 18, the control knob, and/or components or elements thereof, for example metallic materials and/or polymeric materials, are described below.

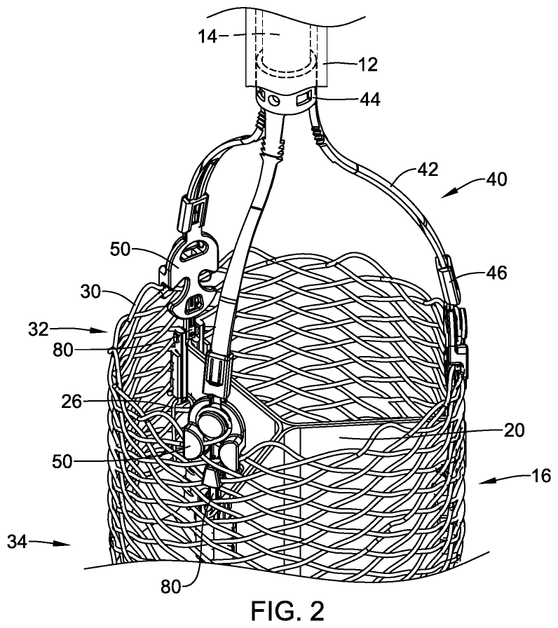

In use, the medical device system 10 may be advanced percutaneously through the vasculature to a position adjacent to an area of interest or a target location. For example, the medical device system 10 may be advanced through the vasculature and across the aortic arch to a position adjacent to a defective aortic valve. Alternative approaches to treat a defective aortic valve and/or other heart valve(s) are also contemplated with the medical device system 10. During delivery, the replacement heart valve implant 16 may be generally disposed in an elongated and low profile “delivery” configuration coupled to the implant delivery device and/or disposed within the outer sheath 12 coupled to and/or distal of the inner catheter 14, as seen in FIG. 1, for example. Once positioned, the outer sheath 12 may be retracted relative to the inner catheter 14, which may be held stationary by the handle 18, and/or the replacement heart valve implant 16 to expose the replacement heart valve implant 16. The replacement heart valve implant 16 may be actuated using the handle 18 and/or the control knob in order to translate the replacement heart valve implant 16 into a generally expanded and larger profile “deployed” configuration (e.g., expanded but still coupled to the implant delivery device and/or the inner catheter 14, as in FIG. 2) suitable for implantation within the anatomy. When the replacement heart valve implant 16 is suitably deployed within the anatomy, the replacement heart valve implant 16 may be released and/or detached from the medical device system 10 and the implant delivery device can be removed from the vasculature, leaving the replacement heart valve implant 16 in place in a “released” configuration to function as, for example, a suitable replacement for the native aortic valve. In at least some interventions, the replacement heart valve implant 16 may be deployed within the native valve (e.g., the native valve is left in place and not excised). Alternatively, the native valve may be removed (such as through valvuloplasty, for example) and the replacement heart valve implant 16 may be deployed in its place as a replacement.

In some embodiments, the inner catheter 14 may be a tubular structure having one or more lumens extending therethrough. For example, in some embodiments, the inner catheter 14 may include one or more of a first lumen, a second lumen, a third lumen, and a fourth lumen. Other configurations are also contemplated. In some embodiments, the one or more lumens may extend along an entire length of the inner catheter 14. Other embodiments are contemplated, however, wherein one or more of the one or more lumens extend along only a portion of the length of the inner catheter 14. In some embodiments, a coupler assembly 40 may be attached to the inner catheter 14 at and/or proximate a distal end of the inner catheter 14, as seen in FIG. 2. In some embodiments, the coupler assembly 40 may releasably couple the replacement heart valve implant 16 to the inner catheter 14. The coupler assembly 40 may include a plurality of coupler arms 42 extending from a coupler ring 44 to the replacement heart valve implant 16. The coupler ring 44 may be fixedly attached to the distal end of the inner catheter 14. The coupler assembly 40 may also include a plurality of collars 46 releasably securing the plurality of coupler arms 42 to the replacement heart valve implant 16, as discussed herein. The plurality of collars 46 may be slidably disposed on and/or over the plurality of coupler arms 42. For example, the coupler assembly 40 may have one collar 46 slidably disposed on and/or over each coupler arm 42. In some embodiments, the plurality of coupler arms 42 may include two coupler arms, three coupler arms, four coupler arms, or another suitable or desired number of coupler arms. For illustrative purposes only, the coupler assembly 40 shown includes three coupler arms 42. Similarly, the plurality of collars 46 may include two collars, three collars, four collars, or another suitable or desired number of collars. For illustrative purposes only, the coupler assembly 40 shown includes three collars 46. Some suitable but non-limiting materials for the coupler assembly 40, the plurality of coupler arms 42, the coupler ring 44, and/or the plurality of collars 46, for example metallic materials or polymeric materials, are described below.

In some embodiments, the medical device system 10 may include at least one actuator element releasably connecting the medical implant to the handle 18. For example, the at least one actuator element may extend from the handle 18 to the replacement heart valve implant 16, the medical implant being disposed at a distal end of the lumen of the outer sheath 12. The at least one actuator element may extend distally from the inner catheter 14 to the replacement heart valve implant 16. In some embodiments, the at least one actuator element may be slidably disposed within and/or may extend slidably through the inner catheter 14. For example, the at least one actuator element may be slidably disposed within one or more of the lumens of the inner catheter 14, and may be used to actuate (i.e., translate axially or longitudinally, and/or expand) the replacement heart valve implant 16 between the “delivery” configuration, the “deployed” configuration, and/or the “released” configuration. In some embodiments, the at least one actuator element may include a plurality of actuator elements, two actuator elements, three actuator elements, four actuator elements, or another suitable or desired number of actuator elements. In some embodiments, the at least one actuator element may be disposed along and/or associated with the plurality of coupler arms 42. For example, one actuator element may be slidably disposed along and/or associated with each coupler arm 42. The at least one actuator element may extend through one collar 46. For example, one actuator element may be slidably disposed within and/or extend through the collar 46 associated with its respective coupler arm 42.

For the purpose of discussion only, the medical device system 10 and/or the replacement heart valve implant 16 of FIG. 2 may be configured to use three actuator elements. In use, a proximal end of each actuator element may be connected to the handle 18, and/or manipulated or otherwise actuated by a user using the handle 18 and/or the control knob, to reversibly shift the replacement heart valve implant 16 from a “delivery” configuration to a “deployed” configuration, and to later shift the medical implant from the “deployed” configuration to a “released” configuration. During the release process for the replacement heart valve implant 16, (e.g., as the replacement heart valve implant 16 is actuated from the “delivery” configuration to the “deployed” configuration to the “released” configuration), the at least one actuator element may be retracted, withdrawn, and/or translated proximally relative to the inner catheter 14, the plurality of coupler arms 42, the plurality of collars 46, and/or the replacement heart valve implant 16. Some suitable but non-limiting materials for the actuator element, for example metallic materials or polymeric materials, are described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the actuator element”, “the leaflets”, “the lumen”, or other features may be equally referred to all instances and quantities beyond one of said feature. As such, it will be understood that the following discussion may apply equally to any and/or all of the components for which there are more than one within the replacement heart valve implant 16 (i.e., the at least one actuator element, the plurality of leaflets, etc.) and/or the medical device system 10, unless explicitly stated to the contrary. Additionally, not all instances of some elements or features may be shown in each figure for clarity.

FIG. 2 illustrates some selected components of the replacement heart valve implant 16. For example, here it can be seen that the replacement heart valve implant 16 may include a plurality of valve leaflets 20 (e.g., bovine pericardial, polymeric, etc.) disposed within an expandable anchor member 30 that is reversibly actuatable between an elongated “delivery” configuration and a shortened and/or expanded “deployed” configuration. In some embodiments, the expandable anchor member 30 may form a tubular structure defining a central longitudinal axis and a lumen extending through the expandable anchor member 30 along, parallel to, coaxial with, and/or coincident with the central longitudinal axis from an inflow end of the expandable anchor member 30 to an outflow end of the expandable anchor member 30. In at least some embodiments, the expandable anchor member 30 may include a first end portion 32 corresponding to the outflow end of the expandable anchor member 30, and a second end portion 34 opposite the first end portion 32, the second end portion 34 corresponding to the inflow end of the expandable anchor member 30.

In some embodiments, the expandable anchor member 30 may be and/or may include an expandable stent having a plurality of struts. In some embodiments, the expandable anchor member 30 may be and/or include a braid formed from one or more interwoven filaments (e.g., a single filament, two filaments, etc.). The expandable anchor member 30 may include a plurality of interstices (e.g., openings) disposed in the expandable anchor member 30. The plurality of interstices (e.g., openings) may pass from an interior of the expandable anchor member 30 to an exterior of the expandable anchor member 30 between adjacent struts and/or filaments. In some embodiments, the expandable anchor member 30 may be self-expanding. In some embodiments, the expandable anchor member 30 may be expanded via mechanical means, using a balloon, or other suitable methods of expansion. Other configurations are also contemplated. Some suitable but non-limiting materials for the expandable anchor member 30, for example metallic materials, polymeric materials, shape memory materials, etc., are described below.

In some embodiments, the replacement heart valve implant 16 may include a locking system attached to the expandable anchor member 30, the locking system being configured to lock the expandable anchor member 30 in the shortened and/or expanded “deployed” configuration and/or the “released” configuration. In some embodiments, the at least one actuator element may be configured to actuate the expandable anchor member 30 and/or the replacement heart valve implant 16 between the elongated “delivery” configuration and the shortened and/or expanded “deployed” configuration and/or the “released” configuration.

In some embodiments, the locking system may comprise an axially movable post member 80, for example at the commissure portions of the valve leaflets 20 (the post member 80 may sometimes be referred to as a portion of a commissure post, which may serve to secure the valve leaflets 20, or the post member 80 may be connected and/or attached to a commissure post), and a locking element 50 configured to slidably receive the post member 80 therein to engage with the locking element 50 and to thereafter secure and/or lock the expandable anchor member 30 and/or the replacement heart valve implant 16 in the shortened and/or expanded “deployed” configuration and/or the “released” configuration.

The locking element 50 may be releasably securable and/or releasably secured to the first end portion 32 of the expandable anchor member 30. In at least some embodiments, the locking element 50 may be releasably securable and/or releasably secured to the first end portion 32 of the expandable anchor member 30 in the elongated “delivery” configuration without using any external elements and/or without any other structure. For example, the locking element 50 may be releasably securable and/or releasably secured to the first end portion 32 of the expandable anchor member 30 without the use of filaments, sutures, welds, bonding agents, or other fixation elements or structures. When the locking element 50 is releasably secured to the first end portion 32 of the expandable anchor member 30, the locking element 50 may be axially and/or circumferentially fixed in position relative to the expandable anchor member 30.

As seen in FIG. 2, the collar 46 may be slidably engaged with and/or disposed over the coupler arm 42 and a portion of the locking element 50. The coupler arm 42 and the locking element 50 may include interfering features that prevent relative axial movement therebetween while the collar 46 is engaged with the coupler arm 42 and the locking element 50. As such, the collar 46 may prevent the coupler arm 42 from disengaging the locking element 50 until the collar 46 is slid proximally along the coupler arm 42 and/or relative to the locking element 50.

The post member 80 may be secured to the second end portion 34 of the expandable anchor member 30 opposite the first end portion 32 of the expandable anchor member 30, using a suture or filament 26 for example, and the post member 80 may be axially movable relative to the expandable anchor member 30 and/or the locking element 50. The post member 80 may be coupled to the plurality of valve leaflets 20 disposed within the expandable anchor member 30. In the elongated “delivery” configuration, the locking element 50 and the post member 80 may be axially spaced apart and disengaged from each other. During deployment of the replacement heart valve implant 16, the post member 80 may be translated axially toward and into engagement with the locking element 50 by the at least one actuator element, which may be releasably engaged with and/or releasably attached to the post member 80.

In at least some embodiments, the replacement heart valve implant 16 may include a plurality of locking systems, as described herein. For example, the replacement heart valve implant 16 may include a plurality of post members 80 and a corresponding plurality of locking elements 50. In some embodiments, the replacement heart valve implant 16 may include a locking system at each commissure, at each commissure post, and/or between each adjacent pair of valve leaflets. Other configurations and correspondences are also contemplated. Some suitable but non-limiting materials for the locking system(s), the locking element 50, and/or the post member 80, for example metallic materials or polymeric materials, are described below.

In some embodiments, the plurality of valve leaflets 20 may be secured to the expandable anchor member 30 at, adjacent to, and/or using (at least in part) corresponding post members 80. In some embodiments, the plurality of valve leaflets 20 may also be secured to the inflow end and/or the second end portion 34 of the expandable anchor member 30. In some embodiments, the plurality of valve leaflets 20 may be coupled and/or secured (i.e., to the post member 80, to the expandable anchor member 30, and/or back to themselves) using one or more sutures, threads, wires, filaments, adhesives, bonding agents, or other suitable elements and/or combinations thereof. In some embodiments, the plurality of valve leaflets 20 may be directly attached to the expandable anchor member 30. In some embodiments, the plurality of valve leaflets 20 may not be directly attached to the expandable anchor member 30.

In some embodiments, the post member 80 and/or the commissure post may be secured and/or attached to the second end portion 34 of the expandable anchor member 30 (e.g., along the interior of the expandable anchor member) with sutures, tethers, filaments, adhesives, or other suitable fastening elements. In some embodiments, the commissure post and/or the post member 80 may include one or more holes or other features provided to aid in securing and/or attaching the commissure post and/or the post member 80 to the second end portion 34 of the expandable anchor member 30. In some embodiments, the locking element 50 may be releasably securable and/or releasably secured to the first end portion 32 of the expandable anchor member 30 axially aligned with the post member 80. In some embodiments, the post member 80 may be axially translatable relative to the locking element 50 generally parallel to the central longitudinal axis of the expandable anchor member 30 when the post member 80 is at least partially disposed within and/or engaged with the locking element 50.

In some embodiments, the expandable anchor member 30 may have a total of three locking elements and three post members attached thereto. Accordingly, one actuator element may be associated with each post member and locking element (e.g., with each locking system), for a total of three actuator elements in the given example(s). Other embodiments are contemplated where fewer or more locking elements, post members, and/or actuator elements may be utilized.

In some embodiments, a seal member (not shown) may be circumferentially disposed on, about, and/or adjacent the exterior of the expandable anchor member 30, and as the term suggests, may help to seal the exterior of the replacement heart valve implant 16 and/or the expandable anchor member 30 within and/or against a target site or area of interest upon deployment, thereby preventing leakage around the replacement heart valve implant 16 and/or the expandable anchor member 30. The seal member may be disposed outside of the lumen extending through the expandable anchor member 30. In some embodiments, the seal member may be coupled and/or secured to the expandable anchor member 30 at one or more locations.

In some embodiments, the seal member may include a plurality of grommets. The plurality of grommets may be attached to, bonded to, and/or at least partially embedded in the seal member. The plurality of grommets may act as reinforcement points for attachment of the seal member to the expandable anchor member 30 using a plurality of lashing sutures. In some embodiments, the plurality of lashing sutures may extend through the plurality of grommets. In some embodiments, the plurality of lashing sutures may attach the seal member to the expandable anchor member 30 at non-consecutive intersections of the struts or filaments.

In some embodiments, the seal member may include one or more layers of polymeric material. Some suitable polymeric materials may include, but are not necessarily limited to, polycarbonate, polyurethane, polyamide, polyether block amide, polyethylene, polyethylene terephthalate, polypropylene, polyvinylchloride, polytetrafluoroethylene, polysulfone, and copolymers, blends, mixtures or combinations thereof. Other suitable polymeric materials are also contemplated, some of which are discussed below.

In some embodiments, the modulus of elasticity may vary and/or be different from layer to layer. In other embodiments, the elongation to break may vary and/or be different from layer to layer. In some embodiments, the seal member may also include a reinforcement, a reinforcing layer, and/or one or more reinforcing members added to the polymeric material prior to curing. The reinforcement, the reinforcing layer, and/or the one or more reinforcing members may comprise a woven or nonwoven fabric and may be positioned within or between the various layers. In some embodiments, the reinforcement, the reinforcing layer, and/or the one or more reinforcing members may be positioned on a radially innermost surface or radially outermost surface of the seal member. In some embodiments, the reinforcement, the reinforcing layer, and/or the one or more reinforcing members may be generally aligned. In some embodiments, the reinforcement, the reinforcing layer, and/or the one or more reinforcing members may be randomly oriented and/or disposed on the seal member. In some embodiments, at least a portion of the seal member may be formed from a fabric material having a polymeric coating. In at least some embodiments, the seal member may be impermeable to fluids and/or blood.

In some embodiments, the seal member may include a reinforcing band coupled to the seal member and/or disposed proximate the second end portion 34 (e.g., the inflow end) of the expandable anchor member 30. In some embodiments, the reinforcing band may be integrally formed with, incorporated into, adhered to, and/or at least partially embedded within the seal member. In some embodiments, the reinforcing band may be formed from a woven or nonwoven fabric material, a textile, or other thin flexible material. The reinforcing band may provide tear resistance in the vicinity of sutures, filaments, or other attachment elements associated with components or aspects of the replacement heart valve implant 16. In at least some embodiments, the seal member and/or the reinforcing band does not pass through interstices in the expandable anchor member 30.

In some embodiments, at least one seal stitch may attach the inflow end of the seal member and/or the reinforcing band to the second end portion 34 (e.g., the inflow end) of the expandable anchor member 30. In some embodiments, the at least one seal stitch may include at least one whip stitch. A whip stitch may sometimes be referred to and/or interchanged with a helical stitch. In some embodiments, the at least one seal stitch may form one or more first helical spirals oriented in a first direction about the second end portion 34 (e.g., the inflow end) of the expandable anchor member 30.

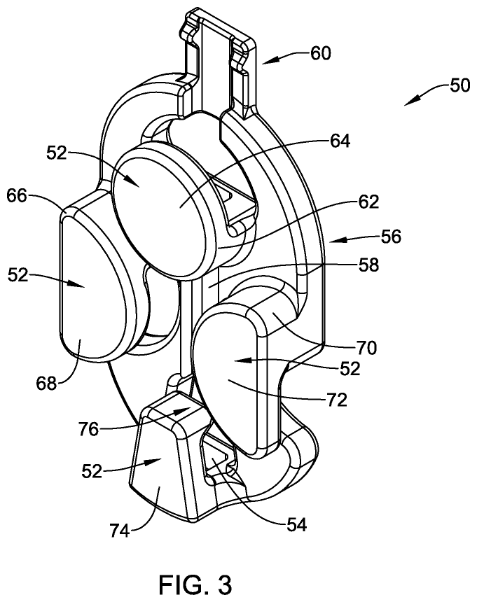

Turning to FIGS. 3-6, which illustrate several views of the locking element 50, the locking element 50 may comprise a plurality of engagement features 52 configured to releasably secure the locking element 50 to the first end portion 32 of the expandable anchor member 30 in the elongated “delivery” configuration and/or the shortened and/or expanded “deployed” configuration without using any external element and/or without any other structure. The locking element 50 may include a plate portion 56 forming a “back” or a “base” of the locking element 50 extending from a first end of the locking element 50 to a second end of the locking element 50. The locking element 50 may include a post passageway 54 configured to axially receive the post member 80. The locking element 50 may include a tongue portion 60 extending axially from the first end of the plate portion 56. The tongue portion 60 may be configured to releasably attach the locking element 50 and/or the replacement heart valve implant 16 to the implant delivery device and/or the coupler arm 42.

The plurality of engagement features 52 may extend laterally from the plate portion 56 in a first direction. The plurality of engagement features 52 may comprise one or more of a head portion 62, a first arm portion 66, a second arm portion 70, and a flap portion 74, as seen in FIG. 3. The head portion 62 may include a head offset 64 laterally offset from and extending generally parallel to the plate portion 56. The first arm portion 66 may include a first arm offset 68 laterally offset from and extending generally parallel to the plate portion 56. The second arm portion 70 may include a second arm offset 72 laterally offset from and extending generally parallel to the plate portion 56. The head portion 62 may be disposed toward the first end of the plate portion 56 relative to the first arm portion 66 and/or the second arm portion 70. The flap portion 74 may be laterally offset from and extending generally parallel to the plate portion 56. The flap portion 74 may include a transverse ridge 76 extending laterally towards the plate portion 56 from the flap portion 74.

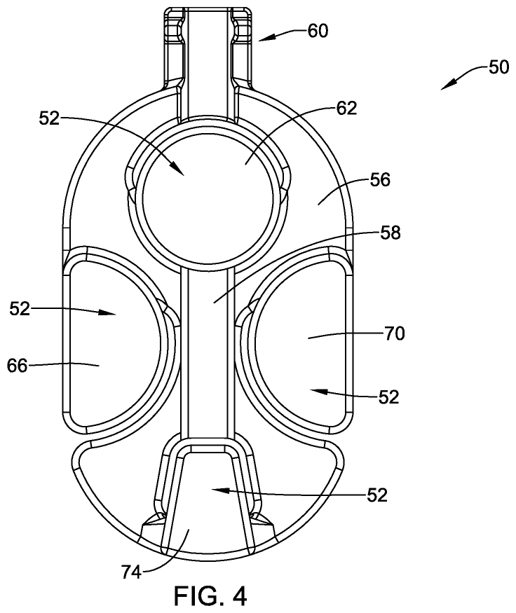

In some embodiments, the plate portion 56 may include an axially-oriented groove 58 formed in the plate portion 56, as seen in FIGS. 3 and 4. The axially-oriented groove 58 may extend from the first end of the locking element 50 to the second end of the locking element 50. The axially-oriented groove 58 may open out in the first direction. The axially-oriented groove 58 may be configured to receive at least a portion of the post member 80 when the expandable anchor member 30 is in the shortened and/or expanded “deployed” configuration. The flap portion 74 and/or the transverse ridge 76 may be aligned with the axially-oriented groove 58. As viewed from the front in FIG. 4, the first arm portion 66 and the second arm portion 70 may be disposed to the left and right, respectively, of the axially-oriented groove 58, the head portion 62, and/or the flap portion 74.

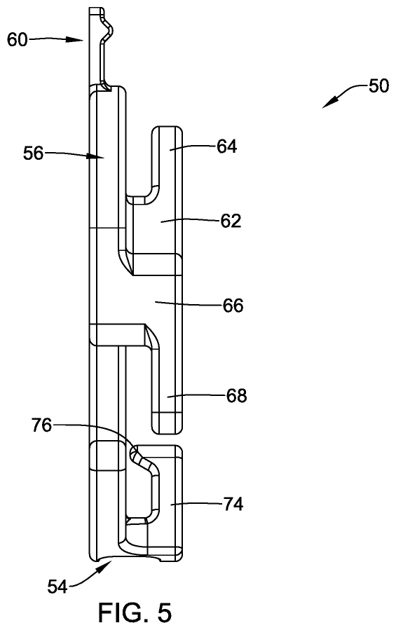

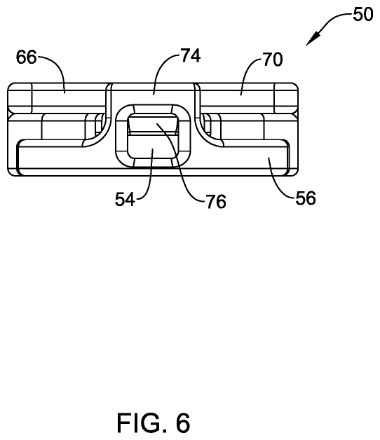

As seen in FIG. 5, the head offset 64 and/or the flap portion 74 may extend axially toward the first end of the locking element 50, and the first arm offset 68 and/or the second arm offset 72 may extend axially toward the second end of the locking element 50. The transverse ridge 76 may extend from the flap portion 74 in a second direction opposite the first direction, and/or laterally towards the plate portion 56, as seen in FIGS. 5 and 6. The post passageway 54 may extend axially from the second end of the locking element 50 toward and/or to the first end of the locking element 50. In at least some embodiments, the post passageway 54 may align with and/or provide access to the axially-oriented groove 58. For example, in some embodiments, the post passageway 54 and the axially-oriented groove 58 may have at least one common and/or coextensive surface.

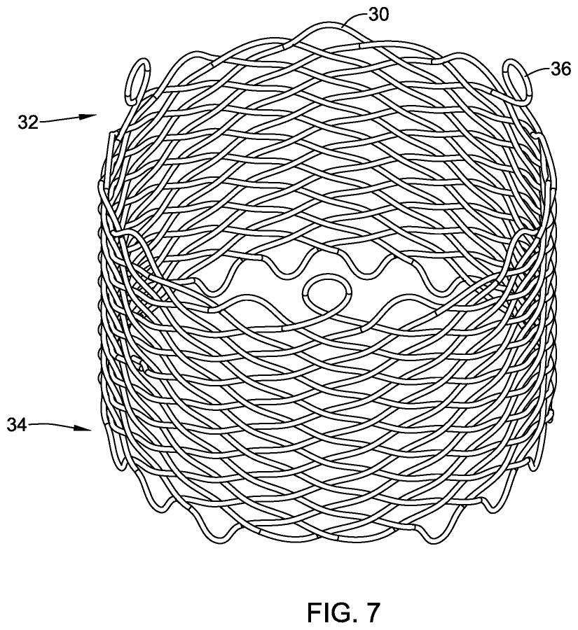

FIG. 7 illustrates aspects of the expandable anchor member 30. The expandable anchor member 30 may include a first end portion 32, which may be and/or may be referred to as an outflow end in some embodiments, and a second end portion 34, which may be and/or may be referred to as an inflow end in some embodiments. In some embodiments, the expandable anchor member 30 may include a loop 36 at and/or extending axially from the first end portion 32 and/or the outflow end of the expandable anchor member 30. In some embodiments, the expandable anchor member 30 may include a plurality of loops 36 at and/or extending axially from the first end portion 32 and/or the outflow end of the expandable anchor member 30, wherein each of the plurality of loops 36 corresponds to and/or is engageable with one locking element 50. In some embodiments, the expandable anchor member 30 may include one or more axially-enlarged crowns extending axially from the first end portion 32 and/or the second end portion 34. In some embodiments, the one or more axially-enlarged crowns may be disposed circumferentially between adjacent loops 36. For example, none of the one or more axially-enlarged crowns may be axially aligned with the loop 36 or the plurality of loops 36. Instead, the one or more axially-enlarged crowns may be circumferentially offset from the loop 36 or the plurality of loops 36. In at least some embodiments, the loop 36 and/or each of the plurality of loops 36 may be a closed loop formed by crossing one filament of the expandable anchor member 30 over itself at an intersection point.

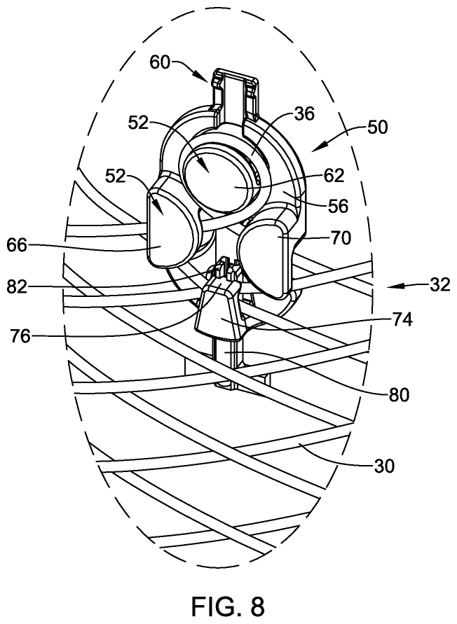

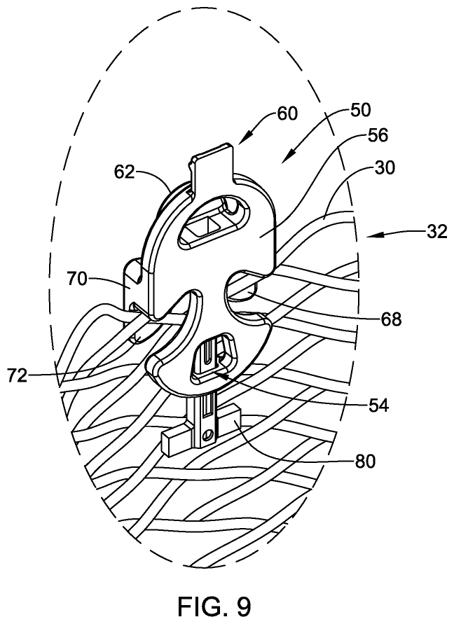

FIGS. 8 and 9 illustrate views of portions of the locking system from the outside and inside of the expandable anchor member 30, respectively. As seen in the figures, the locking element 50 may be configured to engage the post member 80 in the shortened and/or expanded “deployed” configuration. The post member 80 may be secured to the second end portion 34 (e.g., FIG. 2) of the expandable anchor member 30 opposite the first end portion 32 of the expandable anchor member 30. The transverse ridge 76 of the flap portion 74 may be configured to engage a transverse ridge 82 of the post member 80 (e.g., FIG. 8) secured to the second end portion 34 of the expandable anchor member 30 opposite the first end portion 32 in the shortened and/or expanded “deployed” configuration, thereby securing and/or locking the expandable anchor member 30 in the shortened and/or expanded “deployed” configuration.

In some embodiments, the expandable anchor member 30 may surround at least a portion of one or more of the plurality of engagement features 52 when the locking element 50 is releasably secured to the first end portion 32 of the expandable anchor member 30. In some embodiments, the loop 36 at the first end portion 32 of the expandable anchor member 30 may engage at least one of the plurality of engagement features 52. For example, in some embodiments, the loop 36 at the first end portion 32 of the expandable anchor member 30 may surround and/or encircle the head portion 62 of the locking element 50.

In some embodiments, at least a portion of the plate portion 56 may engage an inner surface of the expandable anchor member 30 when the locking element 50 is releasably secured to the first end portion 32 of the expandable anchor member 30. For example, an outwardly facing surface of the plate portion 56 may face, engage with, and/or make contact with the inner surface of the expandable anchor member 30 when the locking element 50 is releasably secured to the first end portion 32 of the expandable anchor member 30. In some embodiments, at least a portion of the plurality of engagement features 52 engages an outer surface of the expandable anchor member 30 when the locking element 50 is releasably secured to the first end portion 32 of the expandable anchor member 30. For example, an inwardly facing surface of the first arm offset 68 and/or an inwardly facing surface of the second arm offset 72 may face, engage with, and/or make contact with the outer surface of the expandable anchor member 30 when the locking element 50 is releasably secured to the first end portion 32 of the expandable anchor member 30. In some embodiments, an inwardly facing portion of the flap portion 74 may face, engage with, and/or make contact with the outer surface of the expandable anchor member 30 when the locking element 50 is releasably secured to the first end portion 32 of the expandable anchor member 30.

In some embodiments, at least a portion of the plurality of engagement features 52 may be disposed radially outward of the expandable anchor member 30. In some embodiments, at least a portion of the plurality of engagement features 52 may extend radially outward through the interstices of the expandable anchor member 30 when the locking element 50 is releasably secured to the first end portion 32 of the expandable anchor member 30. In some embodiments, the head portion 62, the first arm portion 66, the second arm portion 70, and/or the flap portion 74 may be configured to extend exterior of the expandable anchor member 30 when the locking element 50 is releasably secured to the first end portion 32 of the expandable anchor member 30 with the plate portion 56 disposed within and/or radially inward of the expandable anchor member 30. In some embodiments, the head portion 62, the first arm portion 66, the second arm portion 70, and/or the flap portion 74 may be configured to extend exterior of the expandable anchor member 30 when the locking element 50 is releasably secured to the first end portion 32 of the expandable anchor member 30 with the plate portion 56 engaged with and/or in contact with the inner surface of the expandable anchor member 30.

When the locking element 50 is releasably secured to the first end portion 32 of the expandable anchor member 30, in the elongated “delivery” configuration and/or the shortened and/or expanded “deployed” configuration, the plurality of engagement features 52 may prevent the locking element 50 from disengaging from the expandable anchor member 30 without manual manipulation by a physician. For example, while the locking element 50 may be releasably secured to the expandable anchor member 30, the locking element 50 is prevented from releasing and/or disengaging from the expandable anchor member 30 during normal use by the plurality of engagement features 52. It is only through the use of manual manipulation (e.g., twisting and/or tilting of the locking element 50 while spreading apart the filament(s) of the expandable anchor member 30, for example) that the locking element 50 may be disengaged from the expandable anchor member 30. One benefit of this configuration is that no external elements or structures, such as sutures, filaments, tethers, welds, adhesives, etc., are required to secure the locking element 50 to the expandable anchor member 30, but the locking element 50 is still prevented from disengaging from the expandable anchor member 30 during normal use (e.g., implanted and functional) conditions.

Once the replacement heart valve implant 16 has been deployed, and the physician is satisfied with the positioning of the replacement heart valve implant 16, the one or more actuation means disposed within the handle 18 may be activated and/or actuated to axially translate the at least one actuator element proximally. Proximal translation of the at least one actuator element may slide the collar 46 proximally off of the tongue portion 60 of the locking element 50, thereby permitting the locking element 50 to disengage from the coupler arm 42, leaving the replacement heart valve implant 16 disposed within the native heart valve in the “released” configuration. At any time up until the collar 46 has been disengaged from and/or slid proximally off of the tongue portion 60, the locking system may be reversed by advancing the at least one actuator element distally to disengage the post member 80 from the locking element 50, thereby shifting the replacement heart valve implant 16 and/or the expandable anchor member 30 from the shortened and/or expanded “deployed” configuration back towards the elongated “delivery” configuration for repositioning within the native heart valve or removal from the patient.

The materials that can be used for the various components of the medical device system 10 (and/or other systems disclosed herein) and the various elements thereof disclosed herein may include those commonly associated with medical devices. For simplicity purposes, the following discussion refers to the medical device system 10 and/or the replacement heart valve implant 16. However, this is not intended to limit the devices and methods described herein, as the discussion may be applied to other elements, members, components, or devices disclosed herein, such as, but not limited to, the seal member, the reinforcing band, the polymeric coating, the grommets, the sutures, the outer sheath 12, the inner catheter 14, the expandable anchor member 30, the coupler assembly 40, the locking element 50, the post member 80, and/or elements or components thereof.

In some embodiments, the medical device system 10, the implant delivery device, and/or the replacement heart valve implant 16, and/or components thereof, may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the implant delivery device and/or the replacement heart valve implant 16, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device system 10 to achieve the same result.

In some embodiments, a degree of Magnetic Resonance Imaging (MRI) compatibility is imparted into the medical device system 10 and/or the replacement heart valve implant 16 and/or other elements disclosed herein. For example, the medical device system 10 and/or the replacement heart valve implant 16, and/or components or portions thereof, may be made of a material that does not substantially distort the image and create substantial artifacts (i.e., gaps in the image). Certain ferromagnetic materials, for example, may not be suitable because they may create artifacts in an MRI image. The medical device system 10 and/or the replacement heart valve implant 16, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the medical device system 10 and/or the replacement heart valve implant 16 and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the medical device system 10 and/or the replacement heart valve implant 16 and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun-types. The type and denier of the yarn chosen may be selected in a manner which forms a biocompatible and implantable prosthesis and, more particularly, a vascular structure having desirable properties.

In some embodiments, the medical device system 10 and/or the replacement heart valve implant 16 and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A locking element for securing an expandable anchor member in a shortened configuration, comprising:
a plurality of engagement features configured to releasably secure the locking element to a first end portion of the expandable anchor member without using any external elements;
wherein the locking element is configured to engage a post member secured to a second end portion of the expandable anchor member opposite the first end portion in the shortened configuration.

2. The locking element of claim 1, wherein the expandable anchor member surrounds at least a portion of one or more of the plurality of engagement features when the locking element is releasably secured to the first end portion of the expandable anchor member.

3. The locking element of claim 1, wherein the locking element includes a post passageway configured to axially receive the post member.

4. The locking element of claim 1, wherein the locking element includes a plate portion extending from a first end of the locking element to a second end of the locking element.

5. The locking element of claim 4, wherein at least a portion of the plate portion engages an inner surface of the expandable anchor member when the locking element is releasably secured to the first end portion of the expandable anchor member.

6. The locking element of claim 5, wherein at least a portion of the plurality of engagement features engages an outer surface of the expandable anchor member when the locking element is releasably secured to the first end portion of the expandable anchor member.

7. The locking element of claim 5, wherein at least a portion of the plurality of engagement features extends outward through the expandable anchor member when the locking element is releasably secured to the first end portion of the expandable anchor member.

8. The locking element of claim 4, wherein the plate portion includes a groove extending from the first end of the locking element to a second end of the locking element.

9. The locking element of claim 4, wherein each of the plurality of engagement features extends laterally from the plate portion.

10. A replacement heart valve implant, comprising:
an expandable anchor member configured to shift between an elongated configuration and a shortened configuration, the expandable anchor member having a first end portion and a second end portion opposite the first end portion;
a locking element releasably secured to the first end portion of the expandable anchor member by a plurality of engagement features of the locking element without using any external elements; and
a post member secured to the second end portion;
the locking element being configured to secure the expandable anchor member in the shortened configuration when engaged with the post member.

11. The replacement heart valve implant of claim 10, wherein the locking element includes a tongue portion configured to releasably attach the locking element to an implant delivery device.

12. The replacement heart valve implant of claim 10, wherein at least a portion of the plurality of engagement features is disposed outward of the expandable anchor member.

13. The replacement heart valve implant of claim 10, wherein the post member is coupled to a plurality of valve leaflets disposed within the expandable anchor member.

14. The replacement heart valve implant of claim 10, wherein the expandable anchor member includes a loop at the first end portion, the loop engaging at least one of the plurality of engagement features.

15. The replacement heart valve implant of claim 14, wherein the plurality of engagement features prevents the locking element from disengaging from the expandable anchor member without manual manipulation.

16. A locking element for securing an expandable anchor member in a shortened configuration, comprising:
a plate portion;
a tongue portion extending axially from the plate portion; and
a plurality of engagement features extending laterally from the plate portion in a first direction, the plurality of engagement features comprising:
a head portion;
a first arm portion and a second arm portion; and
a flap portion;
wherein the plurality of engagement features is configured to releasably secure the locking element to a first end portion of the expandable anchor member;
wherein the flap portion is configured to engage a post member secured to a second end portion of the expandable anchor member opposite the first end portion in the shortened configuration;
wherein the locking element is releasably securable to the first end portion of the expandable anchor member without any other structure.

17. The locking element of claim 16, wherein the head portion includes a head offset extending generally parallel to the plate portion.

18. The locking element of claim 16, wherein the first arm portion includes a first arm offset extending generally parallel to the plate portion and the second arm portion includes a second arm offset extending generally parallel to the plate portion.

19. The locking element of claim 16, wherein the plate portion includes an axially-oriented groove formed in the plate portion and opening out in the first direction, the axially-oriented groove being configured to receive at least a portion of the post member when the expandable anchor member is in the shortened configuration.

20. The locking element of claim 16, wherein the head portion, the first arm portion, the second arm portion, and the flap portion are configured to extend exterior of the expandable anchor member when the locking element is releasably secured to the first end portion of the expandable anchor member with the plate portion disposed within the expandable anchor member.
